(19) United States
(12) Patent Application Publication
(10) Pub. No.: US 2026/0294968 A1
(43) Pub. Date: Oct. 1, 2026

(54) MODULATION OF B-CELL TRANSLOCATION GENE 1 (BTG1) FOR USE IN ADOPTIVE CELL THERAPY

(71) Applicant: Baylor College of Medicine, Houston, TX (US)

(72) Inventors: Leonid S. Metelitsa, Houston, TX (US); Amy N. Courtney, Houston, TX (US); Xin Xu, Houston, TX (US); Andras Heczey, Houston, TX (US)

(73) Assignee: Baylor College of Medicine, Houston, TX (US)

(21) Appl. No.: 19/106,953

(22) PCT Filed: Aug. 31, 2023
(86) PCT No.: PCT/US2023/031654

§ 371 Date: Feb. 26, 2025

Related U.S. Application Data
(60) Provisional application No. 63/374,447, filed on Sep. 2, 2022.

(51) Int. Cl. A61K 35/17; A61K 35/15; A61K 40/11; A61K 40/15; A61K 40/17; A61K 40/31; A61K 40/32; A61K 40/42; A61K 40/50; A61P 35/00; C07K 14/47; C07K 14/715; C07K 16/00; C12N 5/0783; C12N 5/0786; C12N 15/113; C12N 15/63

(52) U.S. Cl. CPC A61K 35/17; A61K 35/15; A61K 40/11; A61K 40/15; A61K 40/17; A61K 40/31; A61K 40/32; A61K 40/4258; A61K 40/50; A61P 35/00; C07K 14/4738; C07K 14/715; C07K 16/00; C12N 5/0636; C12N 5/0645; C12N 5/0646; C12N 15/113; C12N 15/63; A61K 2239/10; C07K 2317/31; C07K 2319/00; C12N 2310/14; C12N 2310/20; C12N 2310/531; C12N 2510/00; C12N 2830/002; C12N 2830/34

(57) ABSTRACT

Embodiments of the disclosure encompass methods and compositions related to modified cells and uses thereof, wherein the cells are engineered with respect to expression of B-cell translocation gene 1 (BTG1). In specific embodiments, the engineered cells are utilized for adoptive cell therapy for particular disease states. In some embodiments, the engineered cells are immune cells, such as T cells, having reduced expression of BTG1 and are utilized to enhance therapy for cancer and/or infectious disease. In some embodiments, the engineered cells are cells having increased inducible expression of BTG1 and are utilized for therapy for autoimmune disease. In some embodiments, the expression of BTG1 is increased in endogenous cells in an individual with autoimmune disease.

This application claims priority to U.S. Provisional Patent Application Ser. No. 63/374,447, filed Sep. 2, 2022, which is incorporated by reference herein in its entirety.

### I. TECHNICAL FIELD

This disclosure relates at least to the fields of immunology, cell biology, molecular biology, and medicine, including at least cancer medicine.

### II. BACKGROUND

T cell exhaustion is an active process characterized by a progressive loss of effector function and proliferative capacity due to prolonged antigen stimulation that occurs in chronic infections and cancer. Therapeutic immune cells such as T cells or NKT cells engineered to express tumor-specific chimeric antigen receptor (CAR) also undergo exhaustion that limits their antitumor potency and is associated with tumor escape and disease progression or recurrence. Although certain gene expression and epigenetic changes have been implicated in the T exhaustion process, the exact mechanism responsible for hyporesponsiveness of the exhausted cells remains poorly defined.

The present disclosure provides solutions to a long-felt need in the art of improvement of cell therapies.

### BRIEF SUMMARY

Embodiments of the disclosure include methods and compositions for use in therapeutic cells of any kind, including immune effector cells. The therapeutic cells may be tailored and utilized for a particular therapeutic application, such as for cancer treatment, and such cells of this disclosure may be modified to improve one or more activities of the cells. In various embodiments, the present disclosure generally concerns modulation of BTG1 in cells either to (1) prevent or reduce undesirable exhaustion of the cells and/or to control excessive activation with associated toxicity of the therapeutic cells in use for individuals (such as those with cancer); or (2) to modulate endogenous cells of an individual to suppress activity of autoimmune cells (such as for those with an autoimmune disorder).

In some embodiments, expression and/or activity of BTG1 is modulated in cells being used for adoptive cell therapy for any purpose, and the type of modulation is associated with the intended therapeutic use of cells that have been modified accordingly. In various embodiments, the modulation may be to downregulate expression of the endogenous BTG1 gene in a cell to be used for therapy and/or to decrease activity of the BTG1 protein in the cell. In other embodiments, the modulation may be to upregulate expression of endogenous BTG1 gene in a cell, and/or to introduce heterologous BTG1 in the cell (including by overexpression), and/or to increase activity of the BTG1 protein in the cell.

In particular embodiments, a cell for cell therapy is prone to or otherwise has exhaustion, insufficient persistence, insufficient activation, insufficient expansion, insufficient homing, and/or insufficient cytotoxicity. Such cells may be subject to modulation of BTG1 expression and/or activity in order to improve one or more of these characteristics. Some types of immune cells may benefit from modulation of BTG1 expression and/or activity in order to improve one or more of these characteristics. In specific embodiments, expression and/or activity of endogenous BTG1 is reduced in cells to be used for cell therapy, such as for cancer or chronic infectious disease, and such cells may or may not be particular types of immune cells. In various embodiments, T cells of any kind (including CD8 T cell, CD4 T cell, αβ T cells, γ/δ T cells, virus-specific T cells (viral examples including Epstein-Barr virus, cytomegalovirus, BK virus, human herpesvirus, adenovirus, respiratory syncytial virus, Influenza, parainfluenza virus 3, human metapneumovirus, etc.), NKT cells, MAIT cells, cytokine-induced killer cells, NK cells, macrophages, or a mixture thereof, are engineered to have reduced expression and/or activity of endogenous BTG1, and such modulation renders the cells to have reduced or no exhaustion compared to the same type of cells in the absence of such modulation. Such reduction of expression and/or activity of endogenous BTG1 may render the modulated cells to have reduced exhaustion, enhanced persistence, enhanced activation, enhanced expansion, enhanced homing, and/or enhanced cytotoxicity compared to the same type of cells in the absence of such modulation.

In specific embodiments, expression and/or activity of endogenous BTG1 is increased in cells to be used for cell therapy and/or heterologous BTG1 is introduced in the cells, and such cells may be other particular types of cells. In particular embodiments, for a cell for cell therapy it is desirous to be prone to or otherwise have exhaustion, to have insufficient persistence, to have insufficient activation, to have insufficient expansion, to have insufficient homing, and/or to have insufficient cytotoxicity.

In certain embodiments, BTG1 upregulation is useful in endogenous autoimmune cells, such as by a drug that facilitates upregulation of BTG1.

In some embodiments, increased or regulated BTG1 expression can be beneficial for controlling toxicity of therapeutic effector cells in cancer or infections (chronic or acute), such as being used as an “off-switch.” In some embodiments, the BTG1 expression may be controlled by an inducible promoter to inhibit or kill therapeutic cells of any kind encompassed herein when needed, such as at the onset of cytokine storm.

Embodiments of the disclosure include methods of enhancing cell therapy for an individual, comprising the step of reducing expression and/or activity of B-cell translocation gene 1 (BTG1) in the cells for the cell therapy.

Embodiments of the disclosure include methods of enhancing cell therapy for an individual, comprising the step of reducing expression and/or activity of B-cell translocation gene 1 (BTG1) in the cells, wherein the cells are not T cells.

In specific embodiments, the cells are immune cells, stem cells, one or more of their derivatives, or a mixture thereof, optionally when the derivatives are iPSC-derived T, NKT or NK cells. The cells may be CD8 T cells, CD4 T cells, natural killer T (NKT) cells, MAIT cells, γ/δ T cells, Virus-specific T cells, Cytokine-induced killer cells, NK cells, macrophages, or a mixture thereof. In specific embodiments, the cells are modified to express one or more heterologous genes, although in other cases the cells are not modified to express one or more heterologous genes. In specific embodiments, the heterologous gene may comprise one or more engineered receptors, antibodies, cytokines, suicide genes, costimulatory factors regulatory factors, or a combination thereof. The engineered receptor may be an antigen receptor, chemokine receptor, or a cytokine receptor, or the cell may more than one of these types. An antigen receptor may be a chimeric antigen receptor (CAR) or a T cell receptor. A CAR may comprise 1, 2, or more costimulatory domains, such as CD28, 4-1BB, OX40, CD2, DAP10, CD40, ICOS, CD27, TLR, MYD88; 2B4, NKG2D, or a combination thereof. The antigen receptor may target GD2, CD19, GPC3, B7-H3, CD20, BCMA, CD30, CD38, CD5, CD7, HER2, PSMA, mesothelin, EGFR, IL13RA2, or a combination thereof, in specific embodiments. The CAR may comprise one or more activating domains, such as CD35, DAP12, 2B4, or a combination thereof. In specific cases, the cytokine is IL-7, IL-12, IL-15, IL-18, IL-21, IL-23, IL-33, or a combination thereof. In particular embodiments, the antibody is a monospecific antibody, a bispecific antibody, a tri-specific antibody, or a mixture thereof. The antibody may be a bispecific T cell engager or a tri-specific T cell engager. In some embodiments, the reducing step utilizes one or more agents to reduce expression of the endogenous BTG1 gene in the cells. The one or more agents may comprise nucleic acid, peptide, and/or polypeptides. The one or more agents may comprise CRISPR agents, siRNA, shRNA, transposons, or a mixture thereof. In some cases, the reducing step utilizes one or more agents that reduce activity of BTG1 protein in the cells, and the one or more agents may comprise one or more small molecules or one or more antibodies that target BTG1. The method may further comprise the step of administering a therapeutically effective amount of the cells to an individual in need thereof. In some cases, the individual has cancer or acute or chronic infectious disease.

Embodiments of the disclosure may include a plurality of any cells encompassed herein, and the plurality may be comprised in a pharmaceutically acceptable excipient.

In some embodiments, there is provided an engineered non-cancerous cell, said cell engineered to comprise a reduction in expression and/or activity of BTG1, wherein the cell expresses one or more heterologous genes. The heterologous gene may comprise one or more engineered receptors, antibodies, cytokines, suicide genes, costimulatory factors regulatory factors, or a combination thereof. The engineered cell may be an immune cell or a stem cell. The cells in specific embodiments are CD8 T cells, CD4 T cells, NKT cells, MAIT cells, γ/δ T cells, Virus-specific T cells, Cytokine-induced killer cells, NK cells, macrophages, or a mixture thereof. In some embodiments, the reduction in expression was produced by one or more CRISPR agents, siRNA, shRNA, transposons, or a mixture thereof. The reduction in activity may be produced by one or more small molecules. In some embodiments, the engineered receptor is an antigen receptor or a cytokine receptor as described elsewhere herein. In some embodiments, reduction in activity of BTG1 is by one or more small molecules or one or more antibodies that target BTG1.

Embodiments of the disclosure include an engineered CD8 T cell, CD4 T cell, NKT cell, MAIT cell, γ/δ T cell, Virus-specific T cell, Cytokine-induced killer cell, NK cell, macrophage, or a mixture thereof, said cell engineered to comprise a reduction in expression and/or activity of BTG1. The cell may express one or more heterologous genes, such as one or more engineered receptors, antibodies, cytokines, suicide genes, costimulatory factors regulatory factors, or a combination thereof.

Embodiments of the disclosure include methods of treating cancer and/or acute or chronic infectious disease in an individual, comprising the step of administering to the individual a therapeutically effective amount of any one of the plurality of cells of the disclosure. In some embodiments, the plurality of cells comprises NK cells, NK T cells, and/or macrophages comprising a GD2 CAR, a GPC3 CAR, CD19 CAR, and/or a B7-H3 CAR. The cells may be allogeneic or autologous with respect to the individual. In specific embodiments, the acute or chronic infectious disease is human immunodeficiency virus, tuberculosis, herpes, viral hepatitis, or COVID.

Embodiments of the disclosure include methods of treating an autoimmune disease in an individual, comprising the step of administering to the individual a therapeutically effective amount of cells comprising an increase in inducible expression and/or activity of BTG1 and/or comprising the step of administering to the individual a therapeutically effective amount of a drug that increases expression and/or activity of BTG1 in endogenous cells of the individual. In some embodiments, the cells are immune cells, stem cells, one or more of their derivatives, or a mixture thereof. The increase in expression may be from expression of BTG1 on a vector in the cells, and the vector may be an extrachromosomal vector or an integrating vector. In some embodiments, the increase in expression is from introduction of a heterologous promoter in a regulatory region of the endogenous BTG1 gene in the cell. The increase in activity of BTG1 may be from introduction of a small molecule into the cell. The cell may or may not express one or more heterologous genes. In specific cases, the autoimmune disease is Type 1 diabetes, Lupus, Alopecia areata, Autoimmune hemolytic anemia, Autoimmune hepatitis, Dermatomyositis, Glomerulonephritis, Granulomatosis with polyangiitis, Graves' disease Guillain-Barré syndrome. Idiopathic thrombocytopenia purpura, juvenile idiopathic arthritis, Myasthenia gravis, myocarditis, Multiple sclerosis, Pemphigus/pemphigoid, Pernicious anemia, Polyarteritis nodosa, Polymyositis, Primary biliary cirrhosis, Psoriasis, Rheumatoid arthritis, Scleroderma/systemic sclerosis, Sjügren's syndrome, Systemic lupus erythematosus, thyroiditis, uveitis, or Vitiligo. The cells may be allogeneic or autologous with respect to the individual.

Embodiments of the disclosure include an engineered cell, wherein the cell is engineered to have an increase in expression of endogenous BTG1 and/or that comprises a vector that expresses heterologous BTG1. The vector may be an extrachromosomal vector or an integrating vector. The increase in expression may be from introduction of a heterologous promoter in a regulatory region of the endogenous BTG1 gene in the cell. The increase in activity of BTG1 may be from introduction of a small molecule into the cell, and the cell may express one or more heterologous genes.

Embodiments include methods of controlling activity and/or toxicity of a cell therapy, comprising the step of increasing expression and/or activity of BTG1 in the cells of the cell therapy. The increase in expression of BTG1 may be by an inducible promoter. Embodiments may include methods of reducing expression of the endogenous BTG1 gene in the cells, such as by using one or more agents comprise CRISPR agents, miRNA, siRNA, shRNA, transposons, or a mixture thereof, for example.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The disclosure may be better understood by reference to one or more of these drawings in combination with the detailed description of specific embodiments presented herein.

FIGS. 1A-1E: BTG1 expression is elevated in exhausted chimeric antigen receptor (CAR)-natural killer T cells (NKTs). FIG. 1A. Experimental design for repeat tumor co-culture system to induce exhaustion in CAR-NKTs via chronic antigen exposure. Manufactured CAR-NKTs are re-plated with fresh CHLA255 NB tumor cells every five days for multiple cycles. FIG. 1B. Cytotoxic activity of CAR-NKTs during repeated co-culturing at indicated timepoints. FIG. 1C. Uniform Manifold Approximation (UMAP) projections of single-cell RNA sequencing (scRNAseq) results from manufactured CAR-NKT infusion products (IP), CAR-NKTs after five-cycle repeat co-culture with tumor cells (5RcC), and CAR-NKTs isolated from peripheral blood (PB) post-infusion. FIG. 1D. UMAP projection of CAR-NKT gene expression by scRNAseq from pre-infusion and post-5RcC samples. FIG. 1E. Volcano plot demonstrating differentially expressed genes in CAR-NKTs post-5RcC versus pre-infusion products.

FIGS. 2A-2E: Overexpression of BTG1 reduces global RNA expression and proliferative capacity in NKTs: FIG. 2A. Design of retroviral constructs encoding BTG1-green fluorescent protein fusion (BTG1.GFP) or GFP and firefly luciferase (GFP control) control to evaluate the impact of BTG1 overexpression (OE) in NKTs. FIG. 2B. BTG1 expression in NKTs expressing the GFP. BTG1 construct versus wild-type (WT) NKTs by qPCR. FIG. 2C. BTG1 expression in NKTs expressing the GFP. BTG1 construct versus wild-type (WT) NKTs by Western blot. FIG. 2D. Pathway enrichment analysis indicating gene expression programs enriched in BTG1 OE NKTs. FIG. 2E. Fold expansion of BTG1 OE vs GP control NKTs; six independent donors evaluated.

FIGS. 3A-3C. Evaluating the role of BTG1 in regulating the antitumor properties of NKTs. FIG. 3A. BTG1 protein expression as determined by western blot at indicated timepoints in repeat tumor challenge assay (RTC). FIG. 3B. Protein expression of BTG1 measured by western blot after activation with CD3/CD28 specific monoclonal antibodies FIG. 3C. BTG1 mRNA expression as determined by qPCR at indicated timepoints.

FIGS. 4A-4B. BTG1 expression in T cells upon activation: Peripheral blood T cells were stimulated with plate bound CD3/CD28 antibody and cultured in the presence of IL2. FIG. 4A. BTG1 protein expression measured by Western blot at indicated timepoints. FIG. 4B. Absolute number of BTG1 OE vs GFP control T cells after ex vivo culture.

FIGS. 5A-5C. BTG1 KD in GD2-CAR NKTs. MicroRNAs targeting BTG1 and scrambled control were cloned into MMuLV-based gamma-retroviral constructs downstream from the GD2-CAR. FIG. 5A. Retroviral construct designs for BTG1 knockdown. FIGS. 5B, 5C. BTG1 transcript and protein expression in NKTs expressing indicated constructs quantified by qPCR and Western blot, respectively.

FIGS. 6A-6K. BTG1 knockdown (KD) enhances the antitumor activity of GD2-CAR NKTs. NKTs were transduced with retroviral vectors encoding the CAR +/− interleukin-15 (IL15) and/or artificial microRNAs (amiR) specific for BTG1 or a scrambled control. FIG. 6A. Fold expansion of NKTs expressing indicated constructs post-transduction. FIG. 6B. CD62L frequency post-transduction in CAR.15 NKTs with or without BTG1 KD. FIG. 6C. CD62L expression in CAR.15.amiR-BTG1 NKTs gated on CAR+ and CAR− populations. FIG. 6D. Frequency of PD-1+ CAR.15 NKTs with and without BTG1 KD. FIG. 6E. Cytolytic activity of CAR.15.amiR. BTG1 versus scrambled control NKTs against GD2-high CHLA255 and GD2-low CHLA136 NB cell lines assessed at indicated co-culture time-points. FIG. 6F. Residual tumor cell frequencies following five-day co-culture of indicated NKT groups and CHLA255 cells at 1 to 5 E:T ratio. FIG. 6G. CAR-NKT cell fold expansion after 6-cycles co-culture with NB cells. FIG. 6H. Experimental design for in vivo evaluation of CAR-NKT antitumor activity in an aggressive metastatic NB xenograft model. FIG. 6I. Bioluminescence images of tumor-bearing mice at specified timepoints. FIG. 6J. Tumor burden changes based on bioluminescence images over time. FIG. 6K. Kaplan-Meier survival curves for mice in indicated groups. 10 mice per therapeutic group, survival comparison by Gehan-Breslow-Wilcoxon test.

FIGS. 7A and 7B. BTG1 KD enhances the antitumor activity of GD2-CAR T cells. FIG. 7A) GD2-CAR T cell fold expansion after three rounds of co-culture with CHLA255 neuroblastoma (NB) cells (E:T=1:1, N=6, two-tailed paired t-test). 7B) CAR percentage change of T cells before or after three rounds of co-culture with CHLA255 NB cells (N=6, two-tailed paired t-test).

FIGS. 8A-8F. BTG1 KD enhances the antitumor activity of GD2-CAR T cells in an in vivo metastatic NB xenograft model. FIG. 8A) Experimental design for in vivo evaluation of GD2-CAR T cell antitumor activity in an aggressive metastatic NB xenograft model. FIG. 8B) Schemes of retroviral constructs for BTG1 KD and scrambled miRNA control. LTR, long terminal repeat. scFv, single chain variable fragment. H-TM, hinge-transmembrane. FIG. 8C) Bioluminescence images of tumor-bearing mice at specified time points. FIG. 8D) Tumor burden changes based on bioluminescence images in C) over time. FIG. 8E) Kaplan-Meier survival curves for mice in the indicated groups, 10 mice per group, survival comparison by Gehan-Breslow-Wilcoxon test. FIG. 8F) Quantification of human T cells (human CD45+ in total cell populations collected from mouse blood at day 10).

FIG. 9A-9D. BTG1 deletion in T cells increases the frequency of memory T cells. FIG. 9A) Experimental design using CRISPR methodology to KI CD34-Q8 tag at BTG1 locus. FIG. 9B) Representative flow cytometry of CD34-Q8 tag expression in T cells five days after CRISPR KI. FIG. 9C) BTG1 protein expression in T cells by Western blot seven days after CRISPR KI. FIG. 9D) Representative plots and summary showing expression of memory markers CD45RA and CCR7 in T cells with CD34-Q8 tag KI at BTG1 locus and Cas9 only control (N=4).

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

An “autoimmune disease” refers to a disease in which the immune system produces an immune response (for example, a B cell or a T cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

As used herein, a “reduction of expression” or “disruption” or “alteration” of a gene refers to the total or partial elimination or decrease of expression of one or more gene products encoded by the subject gene in a cell, compared to the level of expression of the gene product in the absence of the alteration. Exemplary gene products include mRNA and protein products encoded by the gene. Alteration in some cases is transient or reversible and in other cases is permanent. Alteration in some cases is of a functional or full length protein or mRNA, despite the fact that a truncated or non-functional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of or associated with the gene, such as at the DNA level. Exemplary methods for gene alteration include gene silencing, knockdown, knockout, and/or gene alteration techniques, such as gene editing. Examples include antisense technology, such as RNAi, siRNA, shRNA, and/or ribozymes, which generally result in transient reduction of expression, as well as gene editing techniques which result in targeted gene inactivation or alteration, e.g., by induction of breaks and/or homologous recombination. Examples include insertions, mutations, and deletions. The alterations typically result in the repression and/or complete absence of expression of a normal or “wild type” product encoded by the gene. Exemplary of such gene alterations are insertions, frameshift and missense mutations, deletions, knock-in (including knock-in with a marker, such as a CD34-Q8 tag, GFP, or another selection marker), and knock-out of the gene or part of the gene, including deletions of the entire gene. Such alterations can occur in the coding region, e.g., in one or more exons, resulting in the inability to produce a full-length product, functional product, or any product, such as by insertion of a stop codon. Such alterations may also occur by alterations in the promoter or enhancer or other region affecting activation of transcription, so as to prevent transcription of the gene. Gene alterations include gene targeting, including targeted gene inactivation by homologous recombination.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. Cells may be engineered to express heterologous proteins that are not naturally expressed by the cells, either because the heterologous proteins are recombinant or synthetic or because the cells do not naturally express the proteins. An engineered entity is not found in nature.

The terms “exhausted” or “exhaustion” as used herein refers to immune cells, including T cells, in which the cells become dysfunctional by exhibiting poor effector function, sustained expression of inhibitory receptors, and a transcriptional state distinct from that of functional effector or memory T cells. Exhaustion can occur during chronic infection, autoimmune disease, or cancer, and it can prevent optimal control of infection and tumors, respectively.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

“Natural Killer T (NKT) cells” as used herein may refer to a subset of innate-like T lymphocytes that recognize glycolipids presented by the monomorphic MHC-like molecule CD1d. Unlike T cells, NKTs do not recognize HLA class I or class II molecules. Type I or invariant NKTs express invariant T cell receptor (TCR) α-chain Vα24-Jα18, which in specific embodiments is paired with VβB11. This subset of NKTs can be identified using monoclonal antibody clone 6B11 or by reactivity to a synthetic glycolipid alpha-galactosylceramide. In specific embodiments, the NKT cells express one or more engineered antigen receptors, such as one or more CARs.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” or “pharmaceutically acceptable excipient” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “subject,” as used herein, generally refers to an individual that has or is suspected of having cancer, or in other cases that has or is suspected of having acute or chronic infectious disease and/or autoimmune disease. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer, infectious disease, or autoimmune disease. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies. The subject may be participating in a clinical trial.

As used herein “treatment” or “treating,” includes any beneficial or desirable effect on the symptoms or pathology of a disease or pathological condition, and may include even minimal reductions in one or more measurable markers of the disease or condition being treated, e.g., cancer, infectious disease, or autoimmune disease. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof. Treating may mean alleviation of at least one symptom of the disease or condition.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease. For example, treatment of cancer may involve, for example, a reduction in the size of a tumor, a reduction in the invasiveness of a tumor, reduction in the growth rate of the cancer, or prevention of metastasis. For example, treatment of cancer may also refer to prolonging survival and/or increasing quality of life of a subject with cancer.

### II. General Embodiments

In various embodiments, cell therapies for individuals in need thereof are prepared such that the cells have modulated expression and/or activity of endogenous BTG1 or in other cases have heterologous BTG1 introduced into the cells.

The disclosure encompasses embodiments wherein cellular therapies for a disease are generated based on the disease being treated. For some embodiments, an individual has a medical condition for which cells for therapy for the condition are needed to have persistence and to avoid exhaustion. In specific embodiments wherein cancer or chronic viral infectious disease (such as HIV, chronic viral hepatitis) is being treated (as examples only), it is beneficial for the cells of the cell therapy to avoid exhaustion or have a reduced extent of exhaustion. As provided elsewhere herein, BTG1 plays a role in the exhaustion of NKT and T cells, for example, and its knockdown or knockout or reduced activity is beneficial to the persistence of the cells. Such cells are effective as therapy for cancer or chronic viral infectious disease.

However, in some embodiments wherein, e.g., an autoimmune disease is being treated, it is beneficial for the cells of the cell therapy to have exhaustion or have an increased or accelerated extent of exhaustion.

Embodiments of the disclosure include methods of enhancing cell therapies for an individual, comprising the step of reducing expression and/or activity of BTG1 in the cells for the cell therapy or increasing expression and/or activity of BTG1 in the cells for the cell therapy. In some cases, the cells may or may not be T cells.

Embodiments of the disclosure include methods of producing cells for cell therapy for an individual, comprising the step of reducing expression and/or activity of BTG1 in the cells, optionally wherein the cells are not T cells. In specific embodiments, provided herein are methods of producing cells for cell therapy for an individual, comprising the step of increasing expression and/or activity of BTG1 in the cells or in endogenous cells of the individual.

In specific embodiments, the disclosure provides methods comprising the step of reducing expression and/or activity of BTG1 in the cells, optionally wherein the cells are not T cells. In some embodiments, provided herein are methods comprising the step of increasing expression and/or activity of BTG1 in the cells or in endogenous cells of the individual.

### III. Reduction in Expression and/or Activity of BTG1

In particular embodiments, cells for use in cancer therapy or therapy of acute or chronic infectious disease are in need of having reduced exhaustion or absence of exhaustion compared to, e.g., natural corresponding cells in vivo or cells lacking modification of BTG1 by the hand of man, for example. In specific cases, such cells may be immune cells, including at least T cells of any kind (including CD8 T cell, CD4 T cell, αβ T cells, γ/δ T cells, virus-specific T cells), NKT cells, MAIT cells, cytokine-induced killer cells, NK cells, macrophages, or a mixture thereof. In particular embodiments, the cells are T cells of any kind or NKT cells. In various embodiments, the cells for cancer therapy or acute or chronic infectious disease therapy have modulation of expression of BTG1, the cells for cancer therapy or acute or chronic infectious disease therapy have modulation of activity of BTG1, or both. In specific embodiments, the modulation of expression includes disruption or reduction of expression of the endogenous BTG1 gene to an extent that is greater than in the absence of the modulation. In specific embodiments, the modulation of activity includes reduction of activity of endogenous BTG1 protein to an extent that is greater than in the absence of the modulation.

In particular embodiments, expression of the endogenous BTG1 gene is reduced in cells for cancer therapy or therapy for acute or chronic infectious disease. The expression may be engineered to be reduced by any suitable manner.

In some embodiments, reduction in BTG1 gene expression is carried out by effecting a disruption in the gene, such as a knock-out, insertion, missense or frameshift mutation, such as biallelic frameshift mutation, deletion of all or part of the gene, e.g., one or more exons or portions therefore, and/or knock-in. For example, the altered BTG1 gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the BTG1 gene or a portion thereof.

In some embodiments, the alteration of the expression, activity, and/or function of the BTG1 gene is carried out by disrupting the gene. In some aspects, the gene is modified so that its expression is reduced by at least about or about 20, 30, or 40%, generally at least about or about 50, 60, 70, 80, 90, or 95% as compared to the expression in the absence of the gene modification or in the absence of the components introduced to effect the modification.

In some embodiments, the alteration is transient or reversible, such that expression of the gene is restored at a later time. In other embodiments, the alteration is not reversible or transient, e.g., is permanent.

In some embodiments, gene alteration is carried out by induction of one or more double-stranded breaks and/or one or more single-stranded breaks in the BTG1 gene, typically in a targeted manner. In some embodiments, the double-stranded or single-stranded breaks are made by a nuclease, e.g. an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene alteration is achieved using antisense techniques, such as by RNA interference (RNAi), short interfering RNA (siRNA), short hairpin (shRNA), and/or ribozymes are used to selectively suppress or repress expression of the BTG1 gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct, such as being included with an expression construct that produces a heterologous gene product.

### A. 1ZFPs and ZFNs

In some embodiments, the DNA-targeting molecule includes a BTG1-binding protein such as one or more zinc finger protein (ZFP) or transcription activator-like protein (TAL), fused to an effector protein such as an endonuclease. Examples include ZFNs, TALEs, and TALENs.

In some embodiments, the BTG1-targeting molecule comprises one or more zinc-finger proteins (ZFPs) or domains thereof that bind to the BTG1 gene in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (-1, 2, 3 and 6) on a zinc finger recognition helix. Thus, in some embodiments, the ZFP or ZFP-containing molecule is non-naturally occurring, e.g., is engineered to bind to a target site of choice.

In some embodiments, the BTG1-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed. (See, for example, Sigma-Aldrich catalog numbers CSTZFND, CSTZFN, CTil-1KT, and PZD0020).

### B. TALs, TALEs and TALENs

In some embodiments, the BTG1-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the BTG1 gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., 2013). A library of TALENs targeting 18,740 human protein-coding genes has been constructed (Kim et al., 2013). Custom-designed TALE arrays are commercially available through Cellectis Bioresearch (Paris, France), Transposagen Biopharmaceuticals (Lexington, Ky., USA), and Life Technologies (Grand Island, N.Y., USA). Specifically, TALENs that target CD38 are commercially available (See Gencopoeia, catalog numbers HTN222870-1, HTN222870-2, and HTN222870-3). Exemplary molecules are described, e.g., in U.S. Patent Publication Nos. US 2014/0120622, and 2013/0315884.

In some embodiments the TALEN s are introduced as trans genes encoded by one or more plasmid vectors. In some aspects, the plasmid vector can contain a selection marker which provides for identification and/or selection of cells which received said vector.

### C. RGENs (Crispr/Cas Systems)

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

In other embodiments, the activity of the endogenous BTG1 protein is reduced in the cells for the cancer therapy or acute or chronic infectious disease therapy, and this may be in addition to or alternative to reducing expression of the endogenous BTG1 gene. The protein may be reduced in activity by any suitable manner, such as with one or more small molecules, one or more antibodies, or a combination thereof. Small molecules may be selected for use with the cells for the cancer therapy from a library and based on their ability to reduce activity in vitro, for example. In some embodiments, one or more antibodies that bind BTG1 may inhibit its activity at least in part or in some cases in full. The antibodies may be of any kind, including monoclonal, polyclonal, etc. BTG1 antibodies are commercially available, or they may be generated by standard means.

### IV. Increase in Expression and/or Activity of BTG1

In certain embodiments, the cells of a cell therapy benefit from the increase in expression and/or activity of BTG1 or endogenous cells of a recipient individual have an increase in expression and/or activity of BTG1. In specific embodiments, such cells are those for use in therapy for any one or more autoimmune diseases, as examples. The cells may have an increase in expression and/or activity by any suitable means. In particular embodiments, the cells have an increase in expression of the BTG1 gene, the cells have an increase in activity of the endogenous BTG1 protein, or both, and in particular embodiments the expression may be inducible to avoid potential toxicity. In embodiments wherein the cells have an increase in expression of the protein, it may be from expression of a heterologous BTG1 gene, such as on a vector. In some embodiments, the increase in expression of the BTG1 gene comes from an increase in expression of the endogenous BTG1 gene in the cells, such as upon introduction of a promoter into a regulatory or other region in a position to be able to regulate the endogenous BTG1 gene. For example, one may be able to introduce a constitutive promoter in a regulatory region of the endogenous BTG1 gene, such as the SV40, CMV, UBC, EF1A, PGK promoters.

In cases wherein heterologous BTG1 is introduced into the cell on a vector, one of skill in the art would be well-equipped to construct a vector through standard recombinant techniques. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and so forth.

In specific embodiments, the vector is a multicistronic vector, and in such cases, a single vector may encode the BTG1 gene and one or more heterologous proteins, such as one or more CARs and/or TCRs, a suicide gene, one or more cytokines, etc.

Viral vectors encoding one or more gene products encompassed herein may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention include retroviral vectors, lentiviral vectors, adenoviral vectors, and adeno-associated viral vectors.

### V. Heterologous Proteins

In specific embodiments, any cells of the disclosure are modified to express one or more heterologous proteins. The heterologous proteins may facilitate activity of the cells in any manner, including at least their activation, persistence, expansion, homing, and/or cytotoxicity, in some embodiments. The modulation of the cells to include one or more heterologous proteins may occur before, during, or after the modulation of expression and/or activity of BTG1. In some cases, the one or more heterologous proteins may be introduced on the same or a different vector as one or more agents that result in decreased expression or activity of BTG1 or one or more agents that result in increased expression or activity of BTG1.

### A. Bispecific or Multi-Specific Antibodies

In some embodiments, the cells are modified to express one or more bispecific or multi-specific antibodies, although in other cases the cells do not express the antibodies but the antibodies are utilized in conjunction with the cells.

In cases wherein the cells are modified to express the antibodies, the antibodies may be engagers that bridge a particular immune effector cell with a particular target cell for destruction of the target cell. In certain embodiments, the engineered cells are used with standard T-cell engagers (BiTEs) because in specific embodiments they have been modified to express CD3 that in many cases is the T cell antigen to which the BiTE engager binds. In such cases, the BiTE may also target a cancer antigen or viral antigen that may be tailored to the medical condition of an intended recipient individual. For example, the BiTE may be tailored to bind a cancer antigen that is characteristic of the cancer cells of a cancer of the individual.

In some cases, the cells may be NK cells modified to express (or not to express but instead used in conjunction with) one or more bispecific NK engagers (BiKEs). The BiKE may comprise an antibody that binds a surface protein on an NK cell, including a naturally expressed surface protein on NK cells, and also comprises an antibody that binds a desired target antigen. The BiKE may target the NK cells through an antibody for a NK surface protein, such as CD16, CS1, CD56, NKG2D, NKG2C, DNAM, 2B4, CD2, an NCR, or KIR, for example. In such cases, the BiKE used in the disclosure may also target a cancer antigen or viral antigen that may be tailored to the medical condition of an intended recipient individual. For example, the BiKE may be tailored to bind a cancer antigen that is characteristic of the cancer cells of a cancer of the individual.

### B. Engineered Receptors

In specific embodiments, the cells are engineered to express one or more engineered receptors. In some cases, the engineered receptors may be engineered antigen receptors that target a cancer or viral antigen of any kind. The receptor may be tailored to target a desired antigen based on cells associated with a medical condition of an intended recipient individual.

### 1. Chimeric Antigen Receptors

In some embodiments, the engineered antigen receptor is a chimeric antigen receptor (CAR). The cells may be modified to encode at least one CAR, and the CAR may be first generation, second generation, or third or a subsequent generation, for example. The CAR may or may not be bispecific for two or more different antigens. The CAR may comprise one or more costimulatory domains. Each costimulatory domain may comprise the costimulatory domain of any one or more of, for example, members of the TNFR superfamily, CD28, CD137(4-1BB), CD134 (OX40), DAP10, DAP12, CD27, CD2, CD5, ICAM-1, LFA-1 (CD11a/CD18), Lck, TNFR-I, TNFR-II, Fas, CD30, CD27, NKG2D, 2B4M, CD40, ICOS, TLR, MYD88; 2B4, or combinations thereof, for example. In certain embodiments, the CAR lacks one or more specific costimulatory domains; for example, the CAR may lack 4-1BB and/or lack CD28.

In particular embodiments, the CAR polypeptide in the cells comprises an extracellular spacer domain that links the antigen binding domain and the transmembrane domain, and this may be referred to as a hinge. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha or CD4, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1 and in certain aspects the CAR polypeptide comprises a particular IgG1 hinge amino acid sequence or is encoded by a particular IgG1 hinge nucleic acid sequence.

The transmembrane domain in the CAR may be derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137,CD154, ICOS/CD278, GITR/CD357, NKG2D, and DAP molecules, such as DAP10 or DAP12. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain.

In particular embodiments, the CAR comprises one or more activating domains, such as CD3λ, DAP12, 2B4, or a combination thereof.

In some embodiments, the engineered receptors utilize one or more homing receptors (that can home to a target not necessarily because of a signal release, such as in the event that they utilize adhesion molecules) and/or one or more chemokine receptors. Examples of chemokine receptors include CXC chemokine receptors, CC chemokine receptors, CX3C chemokine receptors and XC chemokine receptors. In specific cases, the chemokine receptor is a receptor for CCR2, CCR3, CCR5, CCR8, CCR7, CXCR3, L-selectin (CD62L) CXCR1, CXCR2, or CX3CR1.

### 2. T Cell Receptors (TCRs)

In some embodiments, the engineered antigen receptors include recombinant TCRs and/or TCRs cloned from naturally occurring T cells. A “T cell receptor” or “TCR” refers to a molecule that contains a variable alpha and beta chains (also known as TCRalpha and TCRbeta, respectively) or a variable gamma and delta chains (also known as TCRgamma and TCRdelta, respectively) and that is capable of specifically binding to an antigen peptide bound to a MHC receptor. In some embodiments, the TCR is in the alpha/beta form.

Typically, TCRs that exist in alpha/beta and gamma/delta forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail. For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the alpha/beta form or gamma/delta form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable .beta. chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the beta-chain can contain a further hypervariability (HV4) region.

In some embodiments, the TCR chains contain a constant domain. For example, like immunoglobulins, the extracellular portion of TCR chains (e.g., alpha-chain, beta-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., alpha-chain constant domain or Cₐ, typically amino acids 117 to 259 based on Kabat, bet.-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the alpha and beta chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (gamma, delta, and epsilon) in mammals and the zeta-chain. For example, in mammals the complex can contain a CD3gamma chain, a CD3delta chain, two CD3epsilon. chains, and a homodimer of CD3zeta chains. The CD3gamma, CD3delta, and CD3epsilon chains are highly related cell surface proteins of the immunoglobulin superfamily containing a single immunoglobulin domain. The transmembrane regions of the CD3gamma, CD3delta, and CD3epsilon chains are negatively charged, which is a characteristic that allows these chains to associate with the positively charged T cell receptor chains. The intracellular tails of the CD3gamma, CD3delta, and CD3epsilon chains each contain a single conserved motif known as an immunoreceptor tyrosine-based activation motif or ITAM, whereas each CD3zeta chain has three. Generally, ITAMs are involved in the signaling capacity of the TCR complex. These accessory molecules have negatively charged transmembrane regions and play a role in propagating the signal from the TCR into the cell. The CD3- and .zeta.-chains, together with the TCR, form what is known as the T cell receptor complex.

In some embodiments, the TCR may be a heterodimer of two chains alpha and beta (or optionally gamma and delta) or it may be a single chain TCR construct. In some embodiments, the TCR is a heterodimer containing two separate chains (alpha and beta chains or gamma and delta chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### C. Cytokines

In some embodiments, the cells are engineered to express one or more heterologous cytokines and/or are engineered to upregulate normal expression of one or more heterologous cytokines. The cells may or may not be transduced or transfected for one or more cytokines on the same vector as other heterologous genes, such as a CAR.

One or more cytokines may be co-expressed from a vector, including as a separate polypeptide from any component of a heterologous gene product. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. cells expressing IL-15 are capable of continued supportive cytokine signaling, which is useful for their survival post-infusion.

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as one or more components of the CD3 complex with or without the TCR complex.

### D. Antigens

The engineered antigen receptors and the antibodies encompassed by the disclosure may target one or more particular antigens. Among the antigens targeted by the antibodies and/or engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens. In specific embodiments, an antigen targeted by an engineered receptor is GD2, CD19, GPC3, and/or B7-H3.

In particular aspects, the antigens include NY-ESO, CD19, EBNA, CD123, HER2, CA-125, TRAIL/DR4, CD20, CD22, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP,-catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRVIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, and LRRN1.

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### E. Suicide Gene

In particular embodiments, a suicide gene is utilized in conjunction with the cell therapy to control its use and allow for termination of the cell therapy at a desired event and/or time. The suicide gene is employed in transduced cells for the purpose of eliciting death for the transduced cells when needed. The cells of the present disclosure that have been modified to harbor one or more vectors (for example) encompassed by the disclosure that may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk:Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), EGFRv3, and Thymidine phosphorylase (TP), as examples.

### VI. Infectious Disease

In some embodiments, cells for cell therapy are modulated to comprise reduction in expression of BTG1, and these cells are subsequently utilized for therapy of one or more chronic or acute infectious diseases. The type of infectious disease may be caused by bacteria, such as with tuberculosis; viruses, such as human immunodeficiency virus (HIV), viral hepatitis, human papillomavirus virus (HPV), or herpes simplex virus (HSV); fungi; or parasites. In some embodiments, also included are chronic diseases having an infectious origin, such as cervical cancer (human papillomavirus—HPV) and liver cancer (hepatitis B and C viruses). In one embodiment, the infectious disease is COVID.

In particular embodiments, an individual with a acute or chronic infectious disease is administered a therapeutically effective amount of cells, such as certain immune cells, in which BTG1 is inducible and/or its activity increased by administration of a drug. In some embodiments, the cells also are modulated to express one or more heterologous proteins, such as engineered antigen receptors.

### VII. Autoimmune Disease

In some embodiments, an individual is in need of therapy for an autoimmune disease, such as one benefiting from cells in which BTG1 is increased in expression and/or activity. In some cases, a therapeutically effective amount of modulated cells in which BTG1 is increased in expression and/or activity (in a level over cells not modulated in the same manner) is administered to an individual having an autoimmune disease, including having one or more symptoms of an autoimmune disease. Expression of the BTG1 is inducible, in specific embodiments, such as to prevent toxicity. In other cases, the expression and/or activity of BTG1 is increased in an individual with autoimmune disease, such as with a drug.

Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis.

### VIII. Administration of Therapeutic Compositions

In various embodiments, the BTG1-modulated cells are administered to an individual in need thereof. Embodiments of the present disclosure concern methods for the use of the compositions comprising BTG1-modulated cells provided herein for treating or preventing a medical disease or disorder (including cancer, acute or chronic infectious disease, or autoimmune disease). The method includes administering to the subject a therapeutically effective amount of the cells, thereby treating or preventing the disease in the subject, including reducing the risk of, reducing the severity of, and/or delaying the onset of the disease. In certain embodiments of the present disclosure, cancer or infection or disease is treated by transfer of a composition comprising the cell population.

Cancers for which embodiments of the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed. Examples of therapies other than those of the present disclosure include surgery, chemotherapy, drug therapy, radiation, hormone therapy, immunotherapy (other than that of the present disclosure), or a combination thereof.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The quantity to be administered, both according to number of treatments and unit dose, depends on the treatment effect desired. An effective dose is understood to refer to an amount necessary to achieve a particular effect. The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. In embodiments wherein the subject being treated has cancer or acute or chronic infectious disease, this can be the amount of modified cells necessary to improve one or more symptoms, including to inhibit advancement of the disease or cause regression of one or more tumors. In embodimetns wherein the subject being treated has an autoimmune disease, this can be the amount of modified cells necessary to inhibit advancement, or to cause regression of an autoimmune disease, or which is capable of relieving one or more symptoms caused by an autoimmune disease, such as pain and/or inflammation. It can be the amount necessary to relieve symptoms associated with inflammation, such as pain, edema and elevated temperature. It can also be the amount necessary to diminish or prevent rejection of a transplanted organ.

The BTG1-modulated cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation may also depend on the route of administration, and the seriousness of the disease or disorder, and should be decided according to the judgment of the practitioner and each patient's circumstances. The therapeutically effective amount of cells may be dependent on the subject being treated, the severity and type of the affliction, and the manner of administration. In some embodiments, doses that could be used in the treatment of human subjects range from at least 1×10⁴, at least 1×10⁵, at least 1×10⁶, at least 1×10⁷, at least 1×10⁸, at least 1×10⁹, or at least 1×10¹⁰ cells/m². The dose may range from 1×10⁴ to 1×10¹⁰, including 1×10⁵ to 1×10¹⁰ or 1×10⁶ to 1×10¹⁰ or 1×10⁷ to 1×10¹⁰ or 1×10⁸ to 1×10¹⁰ or 1×10⁹ to 1×10¹⁰ and so forth. The exact amount of immune cells is readily determined by one of skill in the art based on the age, weight, sex, and physiological condition of the subject. Effective doses can be extrapolated from dose-response curves derived from in vitro or animal model test systems.

The cells may be administered in combination with one or more other therapeutic agents for the treatment of any affliction. Combination therapies would depend on the affliction and can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In specific embodiments, any of the cells of the disclosure may be obtained from suitable storage prior to modulation. The cells in storage may or may not already have modulation of BTG1 expression and/or activity. The cells in storage may or may not already have expression of any one or more heterologous genes. In some embodiments, any of the cells of the disclosure are obtained from the individual in need of the therapy, are modulated ex vivo with respect to BTG1 expression and/or activity, are optionally modulated to express one or more heterologous genes, and are administered back to the individual. In such cases, a step of modulating the cells for BTG1 may or may not precede modulating the cells for the one or more heterologous genes.

### IX. Kits

Certain aspects of the present disclosure also concern kits comprising compositions of the disclosure or compositions to implement methods of the disclosure. In particular embodiments, the kit comprises cells, fresh or frozen, and that may or may not have been pre-activated or expanded. The cells may or may not already express one or more components of the elements encompassed herein, such as reagents utilized in reducing expression or activity of BTG1, increasing expression or activity of BTG1, cells, vectors, buffers, primers, enzymes, salts, and so forth. The kit may comprise one or more reagents for transfection or transduction of cells, including reagents such as vectors that express the component(s), primers for amplification of the component(s), and so forth. In some cases, the cells may or may not also express one or more heterologous proteins as defined herein, and when they do not, the kit may comprise vectors that express the heterologous protein(s), primers for amplification of the heterologous protein(s), and so forth.

Kits may comprise components that may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more, as examples.

### X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of B-Cell Translocation Gene 1 (BTG1) as a Key Driver of Hyporesponsiveness in Exhausted NKT and T Cells and its use for Cancer Immunotherapy

T cell exhaustion is an active process characterized by a progressive loss of effector function and proliferative capacity because of prolonged antigen stimulation that occurs in acute or chronic infections and cancer. Therapeutic immune cells such as T cells or NKT cells engineered to express tumor-specific chimeric antigen receptor (CAR) (as one example) also undergo exhaustion that limits their antitumor potency and is associated with tumor escape and disease progression or recurrence. Although certain gene expression and epigenetic changes have been implicated in the T exhaustion process, the exact mechanism responsible for hypo-responsiveness of the exhausted cells remains poorly defined. To gain an in-depth insight into the mechanism of exhaustion in clinically relevant effector cells, single cell RNA sequencing (scRNAseq) of CAR NKT cells from 12 patients with neuroblastoma (NB) was performed in the course of a phase I clinical trial (NCT03294954). Changes were assessed in CAR-NKT gene expression following their infusion to patients or multiple rounds of tumor cell challenge in vitro (repeat tumor challenge assay (RTC) assay). CAR-NKTs after 5 RTC rounds and peripheral blood CAR-NKTs 2 weeks post-infusion had similar directional changes in their gene expression profiles compared to infusion product CAR-NKTs, exemplified by the acquisition of terminal effector differentiation and loss of the naïve/memory program. These findings support the five-cycle RTC (Repeat Tumor Challenge assay) as a model that closely recapitulates gene expression changes that occur in CAR-NKTs post-infusion in patients. Comparing gene expression profiles of infusion product versus five-cycle RTC CAR-NKTs revealed significant divergence in the two groups, with 2904 differentially expressed genes identified.

A notable gene that was significantly upregulated in the RTC CAR-NKTs was the B-cell translocation gene 1 (BTG1), which promotes mRNA deadenylation and degradation and was recently described as a mediator of quiescence in naïve murine T cells. The role of this gene in T cell exhaustion has not been described. Consistent with the previously reported rapid downregulation of BTG1 in murine naïve T cells in response to TCR stimulation, BTG1 expression was downregulated relative to unstimulated human NKT or naïve T cells in the first four to six hours after TCR stimulation. However, continued TCR stimulation with CD3/CD28-specific monoclonal antibodies led to sustained upregulation of BTG1 in both NKT and T cells as measured at the protein level by western blot. Overexpression of BTG1 in NKTs induced a finite number of differentially expressed genes but resulted in a global decrease in RNA content in the cells and lowered proliferative capacity. The differentially expressed genes did not overlap with previously identified regulators of T cell exhaustion (i.e., TOX), indicating in some embodiments a unique mechanism of hypo-responsiveness mediated by BTG1 in NKT and T cells. NKTs engineered to co-express a tumor-specific CAR and BTG1-specific shRNA eradicated metastatic NB in mice. Therefore, the study revealed an unanticipated role for BTG1 as a critical regulator of NKT and T cell function. In specific embodiments, like naïve T cells, exhausted T cells and NKT cells upregulate BTG1 to induce global mRNA degradation, contributing to the state of quiescence or hypo-responsiveness shared at least by naïve T and exhausted T cells or NKT cells. Finally, BTG1 downregulation strongly enhances antitumor activity of CAR-redirected NKTs, which is useful to guide the rational design of next-generation cancer immunotherapy products, for example.

### Example 2

### The Role of BTG1 in Hyporesponsiveness in Exhausted NKT and T Cells and its use for Cancer Immunotherapy

The present example provides demonstration of BTG1 association with exhausted immune cells and their consequential role in adoptive cell therapy.

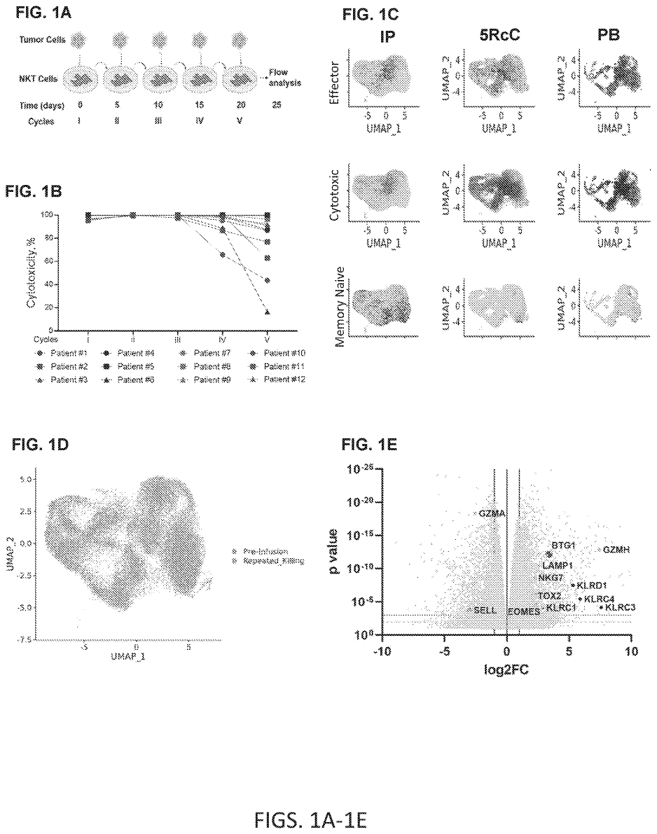

FIG. 1A shows one embodiment of a tumor co-culture system in which CAR-NKTs are re-plated with fresh CHLA255 NB tumor cells every five days for multiple cycles, thereby inducing exhaustion in CAR-NKTs via chronic antigen exposure. The cytotoxic activity of CAR-NKTs during repeated co-culturing at indicated timepoints shows that with time, for many patients the cytotoxicity becomes reduced (FIG. 1B). In FIG. 1C, the Uniform Manifold Approximation and Projection (UMAP) projections of scRNAseq results from infusion products (IP), five-cycle repeat co-culture of infusion products with tumor cells (5RcC), and CAR-NKTs isolated from peripheral blood (PB) post-infusion, and UMAP projections of CAR-NKT gene expression by scRNAseq from pre-infusion and post-5RcC samples are shown in FIG. 1D. FIG. 1E shows a Volcano plot demonstrating differentially expressed genes in CAR-NKTs post-5RcC versus pre-infusion products, including BTG1. BTG1 expression is elevated in exhausted CAR-NKTs.

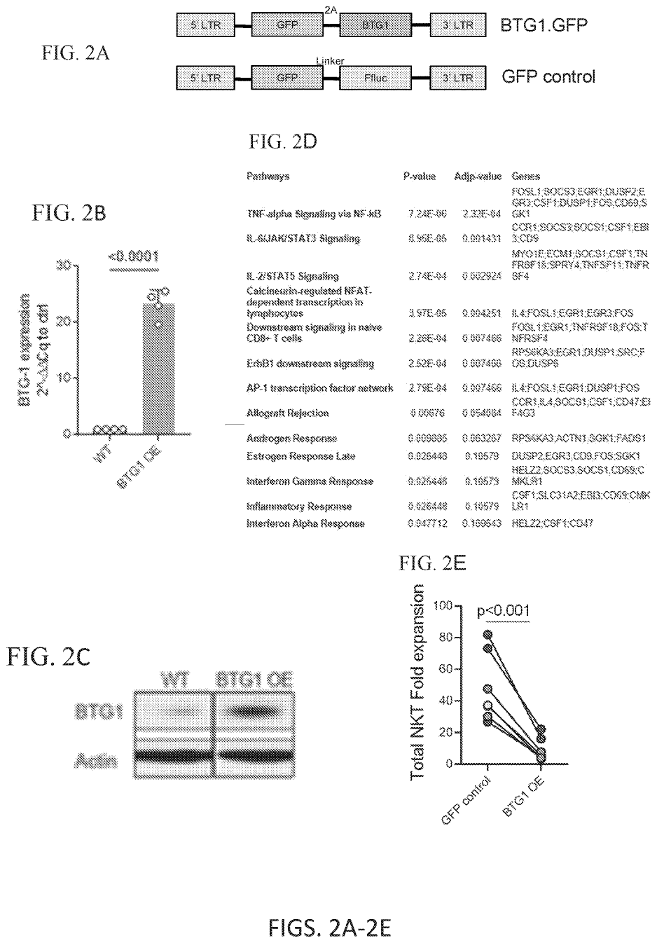

FIG. 2A provides one embodiment of a design of retroviral constructs encoding BTG1.GFP or GFP only as a control to evaluate the impact of BTG1 overexpression (OE) in NKTs. The role of BTG1 in regulating the antitumor properties of NKTs was evaluated. BTG1 protein expression at indicated timepoints by qPCR and Western blot (FIGS. 2B and 2C, respectively). Pathway enrichment analysis indicating gene expression programs enriched in BTG1 OE NKTs (FIG. 2D). The fold expansion of BTG1 OE vs control NKTs in six independent donors was evaluated (FIG. 2E). Overexpression of BTG1 reduces global RNA expression and proliferative capacity in NKTs.

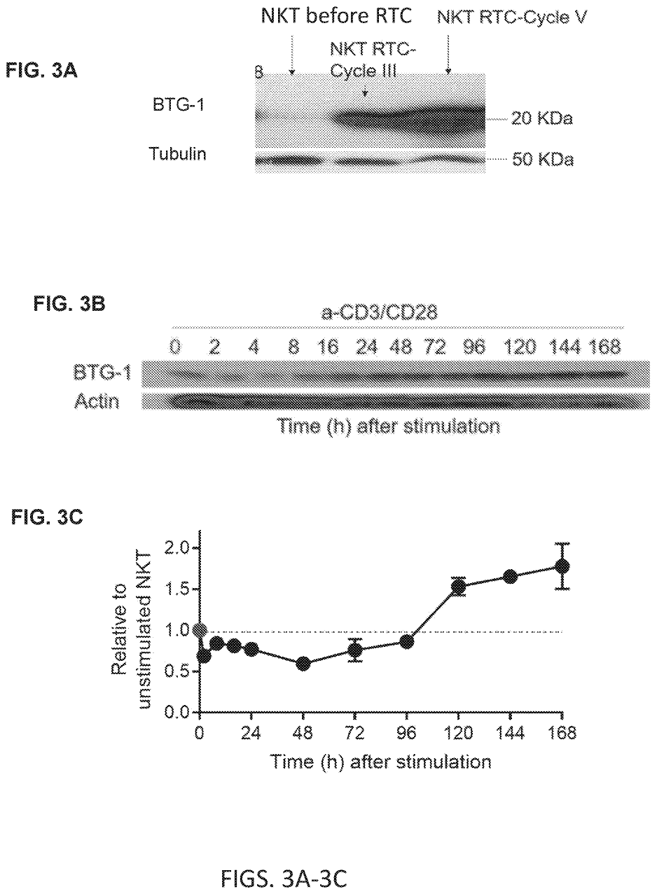

Protein expression of BTG1 was measured by Western blot and BTG1 mRNA was measured by qPCR after activation with CD3/CD28 specific monoclonal antibodies (FIGS. 3B and 3C).

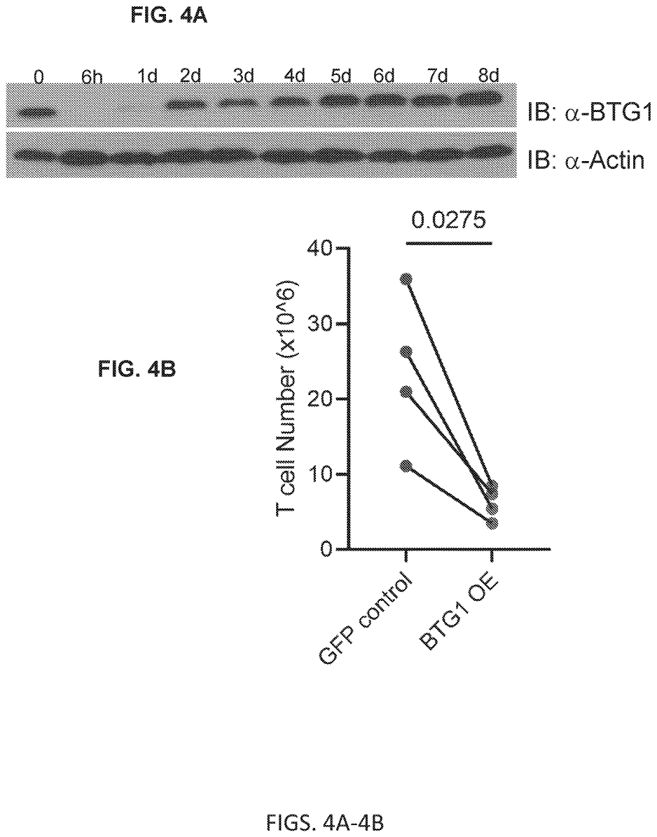

FIGS. 4A-4B demonstrate BTG1 expression in T cells upon activation. Peripheral blood T cells were stimulated with plate-bound CD3/CD28 antibody and cultured in the presence of IL2. BTG1 expression was measured by Western blot at indicated timepoints (FIG. 4A). Absolute number of BTG1 OE vs GFP control T cells were determined after ex vivo culture (FIG. 4B).

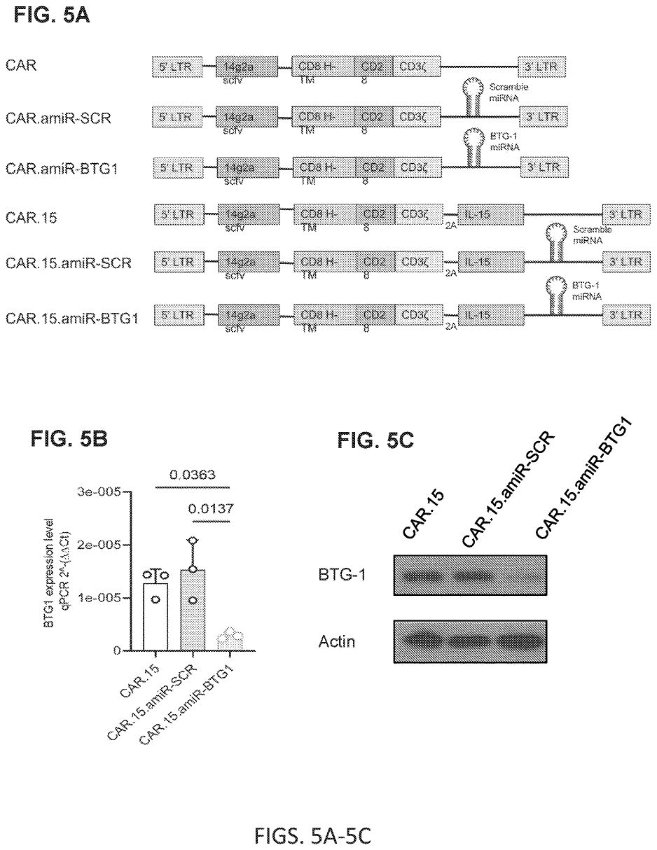

Knockdown of BTG1 in GD2-CAR NKTs is shown in FIG. 5. MicroRNAs targeting BTG1 and scrambled control were cloned into MMuLV-based gamma-retroviral constructs downstream from the GD2-CAR (as merely one example of a CAR). FIG. 5A illustrates example of retroviral construct designs for BTG1 knockdown. The 14g2a scFv for GD2 binding was utilized in all examples of constructs, as was CD8 hinge and transmembrane domain, CD28 costimulatory domain, and CD3zeta. In some cases, IL-15 was utilized for enhancement of activity. In some cases, a control scrambled miRNA (SCR) was included in the construct, while in others a BTG1 miRNA for knockdown was utilized in the construct. FIGS. 5B and 5C show BTG1 transcript level and protein expression in NKTs expressing indicated constructs quantified by qPCR and Western blot, respectively.

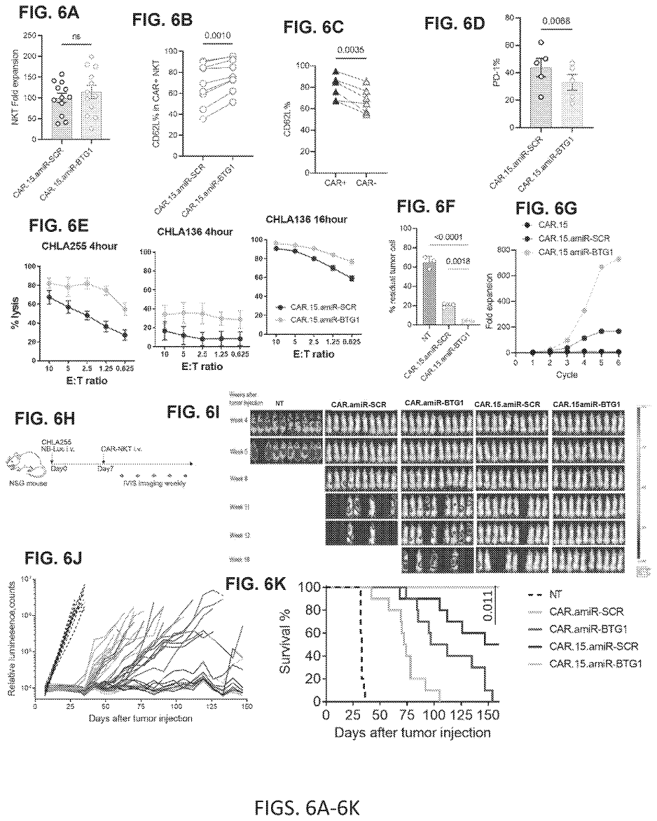

NKTs were transduced with retroviral vectors encoding the CAR +/− IL15 and/or artificial microRNAs (amiR) specific for BTG1 or a scrambled control. Fold expansion of NKTs expressing indicated constructs post-transduction is provided in FIG. 6A. CD62L frequency post-transduction in CAR.15 NKTs with or without BTG1 KD is demonstrated in FIG. 6B. CD62L expression in CAR.15.amiR-BTG1 NKTs gated on CAR+ and CAR− populations is provided in FIG. 6C. FIG. 6D shows the frequency of PD-1+ CAR.15 NKTs with and without BTG1 KD. FIG. 6E provides the cytolytic activity of CAR.15.amiR.BTG1 versus scrambled control NKTs against GD2-high CHLA255 and GD2-low CHLA136 NB cell lines assessed at indicated co-culture time-points. Residual tumor cell frequencies following five-day co-culture are shown of indicated NKT groups and CHLA255 cells at 1 to 5 E:T ratio (FIG. 6F). CAR-NKT cell fold expansion after 6-cycles co-culture with NB cells is provided in FIG. 6G. One exemplary experimental design for in vivo evaluation of CAR-NKT antitumor activity in an aggressive metastatic NB xenograft model is provided in FIG. 6H. Bioluminescence images of tumor-bearing mice at specified timepoints are demonstrated (FIG. 6I). Tumor burden changes based on bioluminescence images over time (FIG. 6J). Kaplan-Meier survival curves for mice in indicated groups, as shown in FIG. 6K. As provided herein, BTG1 knockdown enhances the antitumor activity of GD2-CAR NKTs/

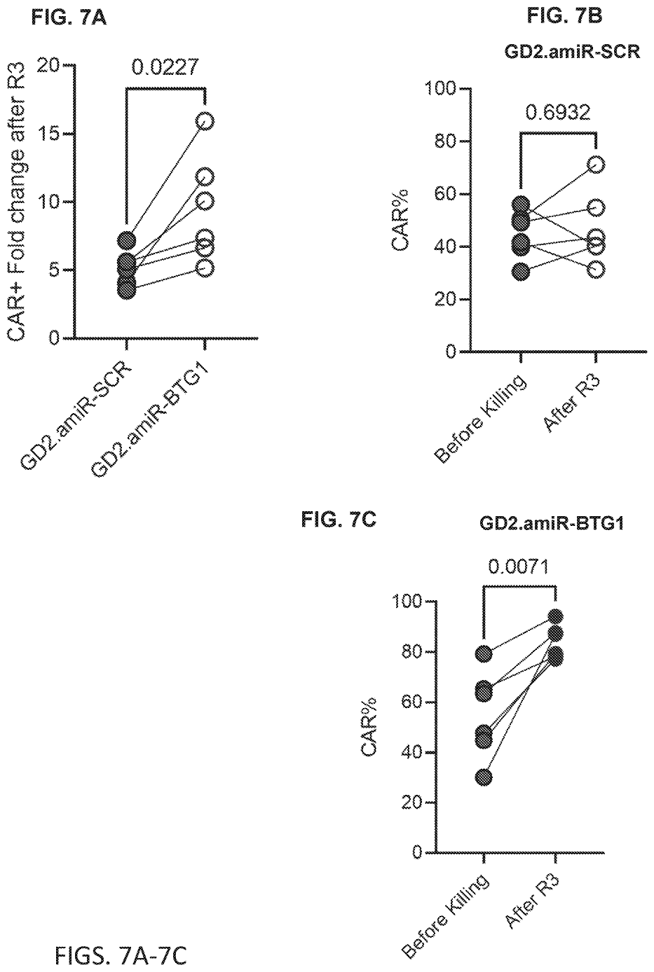

BTG1 knockdown (KD) enhances the antitumor activity of GD2-CAR T cells (as examples) after multiple rounds of in vitro tumor challenge. GD2-CAR T cell fold expansion after three rounds of co-culture with CHLA255 neuroblastoma (NB) cells (E:T=1:1, N=6, two-tailed paired t-test) is provided in FIG. 7A. The CAR % change of T cells is provided in FIG. 7B before or after three rounds of co-culture with CHLA255 NB cells.

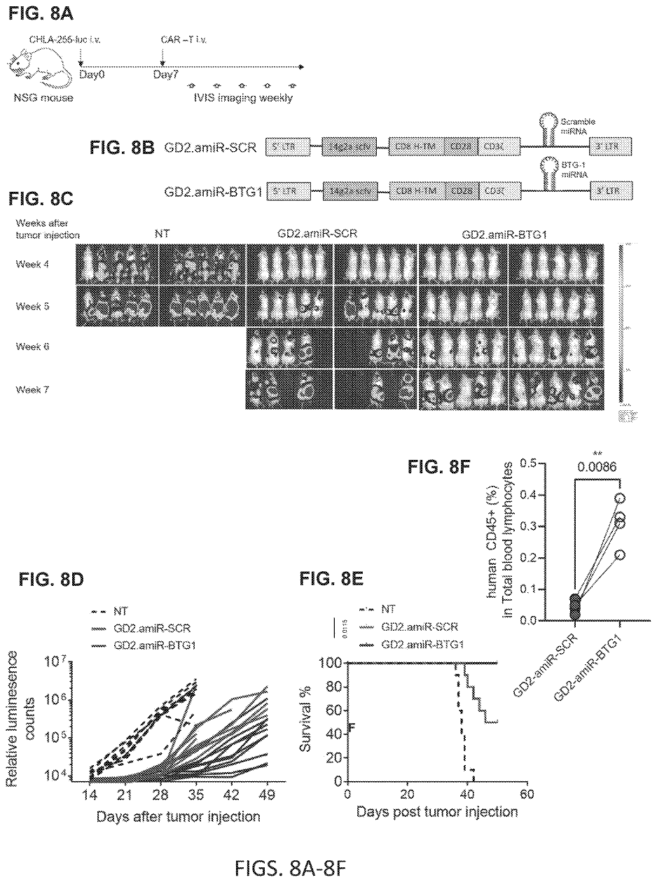

BTG1 KD also enhances the antitumor activity of GD2-CAR T cells in an aggressive in vivo metastatic NB xenograft model. FIG. 8A shows one example of an experimental design for in vivo evaluation of GD2-CAR T cell antitumor activity in the model. Examples of schemes of retroviral constructs for BTG1 KD and scrambled miRNA control (see also FIG. 5A). FIG. 8C demonstrates bioluminescence images of tumor-bearing mice at weeks 4, 5, 6, and 7. Tumor burden changes based on bioluminescence images in FIG. 8C are graphically shown in FIG. 8D, and Kaplan-Meier survival curves for the mice are also provided (FIG. 8E). Quantification of human T cells (human CD45+ in total cell populations collected from mouse blood at day 10) are provided (FIG. 8F).

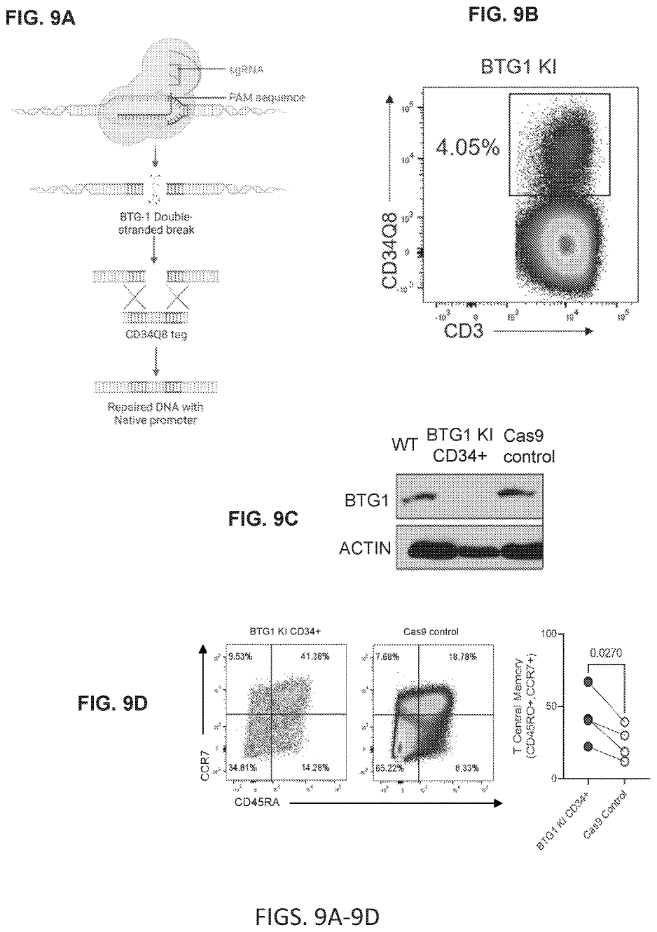

CRISPR Cas9-mediated BTG1 deletion with marker (CD34) knock-in (KI) at BTG1 locus in T cells increases the frequency of memory T cells. FIG. 9A shows one example of an experimental design using CRISPR methodology to KI CD34-Q8 tag at BTG1 locus. Representative flow cytometry of CD34-Q8 tag expression in T cells five days after CRISPR KI is shown. FIG. 9C demonstrates BTG1 protein expression in T cells by Western blot seven days after CRISPR KI. Representative plots and summary showing expression of memory markers CD45RA and CCR7 in T cells with CD34-Q8 tag KI at BTG1 locus with a Cas9 only control are provided in FIG. 9D.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of enhancing cell therapy for an individual, comprising the step of reducing expression and/or activity of B-cell translocation gene 1 (BTG1) in the cells for the cell therapy.

2. A method of enhancing cell therapy for an individual, comprising the step of reducing expression and/or activity of B-cell translocation gene 1 (BTG1) in the cells, wherein the cells are not T cells.

3. The method of claim 1, wherein the cells are immune cells, stem cells, one or more of their derivatives, or a mixture thereof, optionally when the derivatives are iPSC-derived T, NKT or NK cells.

4. The method of claim 1 or 3, wherein the cells are CD8 T cells, CD4 T cells, natural killer T (NKT) cells, MAIT cells, γ/δ T cells, Virus-specific T cells, Cytokine-induced killer cells, NK cells, macrophages, or a mixture thereof.

5. The method of any one of claims 1-4, wherein the cells are modified to express one or more heterologous genes.

6. The method of claim 5, wherein the heterologous gene comprises one or more engineered receptors, antibodies, cytokines, suicide genes, costimulatory factors regulatory factors, or a combination thereof.

7. The method of claim 6, wherein the engineered receptor is an antigen receptor or a cytokine receptor.

8. The method of claim 7, wherein the antigen receptor is a chimeric antigen receptor (CAR) or a T cell receptor.

9. The method of claim 8, wherein the CAR comprises 1, 2, or more costimulatory domains.

10. The method of claim 9, wherein the costimulatory domains comprise CD28, 4-1BB, OX40, CD2, DAP10, CD40, ICOS, CD27, TLR, MYD88; 2B4, NKG2D, or a combination thereof.

11. The method of any one of claims 7-10, wherein the antigen receptor targets GD2, CD19, GPC3, and/or B7-H3.

12. The method of any one of claims 8-11, wherein the CAR comprises one or more activating domains.

13. The method of claim 12, wherein the one or more activating domains comprises CD3λ, DAP12, 2B4, or a combination thereof.

14. The method of any one of claims 6-13, wherein the cytokine is IL-7, IL-12, IL-15, IL-18, IL-21, IL-23, IL-33, or a combination thereof.

15. The method of any one of claims 6-14, wherein the antibody is a monospecific antibody, a bispecific antibody, a tri-specific antibody, or a mixture thereof.

16. The method of any one of claims 6-15, wherein the antibody is a bispecific T cell engager or a trispecific T cell engager.

17. The method of any one of claims 1-16, wherein the reducing step utilizes one or more agents to reduce expression of the endogenous BTG1 gene in the cells.

18. The method of claim 17, wherein the one or more agents comprise nucleic acid, peptide, and/or polypeptides.

19. The method of claim 17 or 18, wherein the one or more agents comprise CRISPR agents, miRNA, siRNA, shRNA, transposons, or a mixture thereof.

20. The method of any one of claims 1-19, wherein the reducing step utilizes one or more agents that reduce activity of BTG1 protein in the cells.

21. The method of claim 20, wherein the one or more agents comprise one or more small molecules or one or more antibodies that target BTG1.

22. The method of any one of claims 1-21, further comprising the step of administering a therapeutically effective amount of the cells to an individual in need thereof.

23. The method of claim 22, wherein the individual has cancer.

24. The method of any one of claims 1-17, wherein the reducing step comprises knock-in or knockdown or knockout of the endogenous BTG1 gene in the cells.

25. The method of claim 24, wherein the knock-in comprises knock-in of a detectable marker.

26. A plurality of cells produced by the method of any one of claims 1-25.

27. The plurality of cells of claim 26, wherein the plurality is comprised in a pharmaceutically acceptable excipient.

28. An engineered non-cancerous cell, said cell engineered to comprise a reduction in expression and/or activity of BTG1, wherein the cell expresses one or more heterologous genes.

29. The cell of claim 28, wherein the heterologous gene comprises one or more engineered receptors, antibodies, cytokines, suicide genes, costimulatory factors regulatory factors, or a combination thereof.

30. The cell of claim 28 or 29, wherein the engineered cell is an immune cell or a stem cell.

31. The cell of any one of claims 28-30, wherein the cell is a cells are CD8 T cells, CD4 T cells, natural killer T (NKT) cells, NKT cells, MAIT cells, γ/δ T cells, Virus-specific T cells, Cytokine-induced killer cells, NK cells, macrophages, or a mixture thereof.

32. The cell of any one of claims 28-31, wherein the reduction in expression was produced by one or more CRISPR agents, siRNA, shRNA, transposons, or a mixture thereof.

33. The cell of any one of claims 28-32, wherein the reduction in activity was produced by one or more small molecules.

34. The cell of claim 29, wherein the engineered receptor is an antigen receptor or a cytokine receptor.

35. The cell of claim 34, wherein the antigen receptor is a CAR or a T cell receptor.

36. The cell of claim 35, wherein the CAR comprises 1, 2, or more costimulatory domains.

37. The cell of claim 36, wherein the costimulatory domains comprise CD28, 4-1BB, OX40, CD2, DAP10, CD40, ICOS, CD27, TLR, MYD88; 2B4, NKG2D, or a combination thereof.

38. The cell of any one of claims 34-37, wherein the antigen receptor targets GD2, CD19, GPC3, and/or B7-H3.

39. The cell of any one of claims 35-38, wherein the CAR comprises one or more activating domains.

40. The cell of claim 39, wherein the one or more activating domains comprises CD35, DAP12, 2B4, or a combination thereof.

41. The cell of any one of claims 28-40, wherein the cytokine is IL-7, IL-12, IL-15, IL-18, IL-21, IL-23, IL-33, or a combination thereof.

42. The cell of any one of claims 29-41, wherein the antibody is a monospecific antibody, a bispecific antibody, or a tri-specific antibody.

43. The cell of any one of claims 29-42, wherein the antibody is a bispecific T cell engager or a trispecific T cell engager.

44. The cell of any one of claims 28-43, wherein reduction in activity of BTG1 is by one one or more small molecules or one or more antibodies that target BTG1.

45. A plurality of the cells of any one of claims 28-44.

46. The plurality of cells of claim 45, wherein the plurality is comprised in a pharmaceutically acceptable excipient.

47. An engineered CD8 T cell, CD4 T cell, NKT cell, MAIT cell, γ/δ T cell, Virus-specific T cell, Cytokine-induced killer cell, NK cell, macrophage, or a mixture thereof, said cell engineered to comprise a reduction in expression and/or activity of BTG1.

48. The cell of claim 47, wherein the cell expresses one or more heterologous genes.

49. The cell of claim 48, wherein the heterologous gene comprises one or more engineered receptors, antibodies, cytokines, suicide genes, costimulatory factors regulatory factors, or a combination thereof.

50. A plurality of the cells of any one of claims 47-49.

51. The plurality of cells of claim 50, wherein the plurality is comprised in a pharmaceutically acceptable excipient.

52. A method of treating cancer and/or acute or chronic infectious disease in an individual, comprising the step of administering to the individual a therapeutically effective amount of any one of the plurality of cells of claim 26, 27, 45, 46, 50, or 51.

53. The method of claim 52, wherein the plurality of cells comprises NK cells, NK T cells, and/or macrophages comprising a GD2 CAR, a GPC3 CAR, and/or a B7-H3 CAR.

54. The method of claim 52 or 53, wherein the cells are autologous with respect to the individual.

55. The method of claim 52 or 53, wherein the cells are allogeneic with respect to the individual.

56. The method of any one of claims 52-55, wherein the infectious disease is human immunodeficiency virus, tuberculosis, herpes, viral hepatitis, or COVID.

57. A method of treating an autoimmune disease in an individual, comprising the step of administering to the individual a therapeutically effective amount of cells comprising an increase in inducible expression and/or activity of BTG1 and/or comprising the step of administering to the individual a therapeutically effective amount of a drug that increases expression and/or activity of BTG1 in endogenous cells of the individual.

58. The method of claim 57, wherein the cells are immune cells, stem cells, one or more of their derivatives, or a mixture thereof.

59. The method of claim 57 or 58, wherein the increase in expression is from expression of BTG1 on a vector in the cells.

60. The method of claim 59, wherein the vector is an extrachromosomal vector.

61. The method of claim 59, wherein the vector is an integrating vector.

62. The method of any one of claims 57-61, wherein the increase in expression is from introduction of a heterologous promoter in a regulatory region of the endogenous BTG1 gene in the cell.

63. The method of any one of claims 57-62, wherein the increase in activity of BTG1 is from introduction of a small molecule into the cell.

64. The method of any one of claims 57-63, wherein the cell expresses one or more heterologous genes.

65. The method of any one of claims 57-64, wherein the autoimmune disease is Type 1 diabetes, Lupus, Alopecia areata, Autoimmune hemolytic anemia, Autoimmune hepatitis, Dermatomyositis, Glomerulonephritis, Granulomatosis with polyangiitis, Graves' disease Guillain-Barré syndrome. Idiopathic thrombocytopenia purpura, juvenile idiopathic arthritis, Myasthenia gravis, myocarditis, Multiple sclerosis, Pemphigus/pemphigoid, Pernicious anemia, Polyarteritis nodosa, Polymyositis, Primary biliary cirrhosis, Psoriasis, Rheumatoid arthritis, Scleroderma/systemic sclerosis, Sjögren's syndrome, Systemic lupus erythematosus, thyroiditis, uveitis, or Vitiligo.

66. The method of any one of claims 57-65, wherein the cells are autologous with respect to the individual.

67. The method of any one of claims 57-65, wherein the cells are allogeneic with respect to the individual.

68. An engineered cell, wherein the cell is engineered to have an increase in expression of endogenous BTG1 and/or that comprises a vector that expresses heterologous BTG1.

69. The cell of claim 68, wherein the vector is an extrachromosomal vector.

70. The cell of claim 68, wherein the vector is an integrating vector.

71. The cell of any one of claims 68-70, wherein the increase in expression is from introduction of a heterologous promoter in a regulatory region of the endogenous BTG1 gene in the cell.

72. The cell of any one of claims 68-71, wherein the increase in activity of BTG1 is from introduction of a small molecule into the cell.

73. The cell of any one of claims 68-72, wherein the cell expresses one or more heterologous genes.

74. A plurality of cells of the cells of any one of claims 68-73.

75. The plurality of cells of claim 74, wherein the plurality is comprised in a pharmaceutically acceptable excipient.

76. A method of controlling activity and/or toxicity of a cell therapy, comprising the step of increasing expression and/or activity of BTG1 in the cells of the cell therapy.

77. The method of claim 76, wherein the increase in expression of BTG1 is by an inducible promoter.
